(19) United States
(12) Patent Application Publication
(10) Pub. No.: US 2026/0108152 A1
(43) Pub. Date: Apr. 23, 2026

(54) VISION SCREENING DEVICE

(71) Applicant: Welch Allyn, Inc., Skaneateles Falls, NY (US)

(72) Inventors: Matthew David Mullin, Memphis, NY (US); Scott A. Martin, Camillus, NY (US); David L. Kellner, Baldwinsville, NY (US); Melissa R. Stancato, Syracuse, NY (US); Joshua F. Hess, Moravia, NY (US); Vivian L. Hunter, Auburn, NY (US); Timothy Bennett, Skaneateles Falls, NY (US)

(21) Appl. No.: 19/339,102

(22) Filed: Sep. 24, 2025

Related U.S. Application Data
(60) Provisional application No. 63/709,024, filed on Oct. 18, 2024.

(51) Int. Cl. A61B 3/14; A61B 3/00; A61B 3/032; A61B 3/11

(52) U.S. Cl. CPC A61B 3/145; A61B 3/0008; A61B 3/032; A61B 3/112

(57) ABSTRACT

A vision screening device for administering vision screening tests to a patient, and in particular vision screening test(s) to determine the presence of diseases and/or abnormalities in the eye(s) of the patient, is described herein. The vision screening device includes emitters for emitting near-infrared light and white light from a patient-facing side and includes a sensor to receive reflected light and energy for performing vision screening tests. The received reflected energy comes into ana emission surface and is reflected by a beam splitter to a sensor. The vision screening device includes a first display for displaying digital objects for a vision test and a second display for a clinician to interact. The vision screening device further includes a sensor to aid in proper positioning of a patient during screening.

### CROSS-REFERENCE TO RELATED APPLICATIONS

This application claims priority to U.S. Provisional Application No. 63/709,024, titled “VISION SCREENING DEVICE”, filed Oct. 18, 2024, which is hereby incorporated by reference in its entirety.

### TECHNICAL FIELD

This application is directed to medical equipment. In particular, this application is directed to a vision screening device, and associated systems and methods, for detection and assessment of diseases and disorders of the eye.

### BACKGROUND

Vision screening typically includes screening for diseases of the eye. Such screening may include a plurality of both visible light, object recognition, color recognition, and infrared light tests to diagnosis a wide variety of potential eye diseases. For example, a transillumination test such as the Brückner red reflex test may be performed. During the red reflex test, the clinician illuminates the eye of the patient with visible light using an ophthalmoscope and examines the color and other characteristics of the light reflected back by the choroid and the retinal surfaces of the eye. Various diseases and abnormalities of the eyes can be detected using this test, such as corneal or media opacities, cataracts, and retinal abnormalities including tumors and retinoblastoma.

In addition, vision screening typically also includes one or more tests to determine various deficiencies associated with the patient's eyes. Such vision tests may include, for example, refractive error tests, accommodation tests, visual acuity tests, color vision screening and the like. Some of the vision screening tests require the use of infrared or near-infrared imaging, while other tests may require imaging under visible light, and/or a display screen to show content to the patient. However, ophthalmic testing devices such as a phoropter, autorefractor and photo-refractors, may only provide the capability to perform a limited range of tests. It would be advantageous to be able to screen for most vision problems and diseases using a single integrated device. Furthermore, due to the number of tests, it may be additional advantageous to be able to conduct a suite of test without repositioning the patient or recalibrating the machine. Current devices may require different distances to accurately conduct each test, resulting in movement of either the patient or the device between test and reducing the overall efficiency of the device.

The various examples of the present disclosure are directed toward overcoming one or more of the deficiencies noted above.

### SUMMARY

In an example of the present disclosure, a vision screening device includes a radiation source configured to emit radiation of a first wavelength, a white light source, a sensor configured to capture radiation reflected by an eye of a patient and further configured to capture a color image of the eye of the patient, and an optional distance sensor. The Vision screening device includes a first display disposed on a patient-facing side of the vision screening device and a second display disposed on a user-facing side of the vision screening device opposite the patient-facing side. The vision screening device further includes one or more processors and one or more non-transitory computer-readable media having instructions stored thereon that, when executed by the processor cause one or more processors to perform operations. The operations include determining a distance to the patient, providing, via the second display, directions to reach a predetermined distance to the patient, and displaying, via the first display, one or more digital objects for a vision test. The operations also include causing the radiation source to emit radiation of the first wavelength towards the patient, causing the sensor to capture first sensor data associated with a portion of the radiation reflected by the eye of the patient, causing the white light source to illuminate the eye of the patient, and causing the sensor to capture second sensor data associated with a portion of white light reflected by the eye of the patient. The vision screening device is further configured to determine, based at least in part on the first sensor data and the second sensor data, an output indicative of a condition associated with the eye.

Implementations may include one or more of the following features. The first wavelength is in a near-infrared (NIR) band of electromagnetic spectrum. The vision screening device may include a beam splitter positioned adjacent the patient-facing side, the beam splitter configured to direct reflected radiation towards the sensor. The vision screening device may include an emitter board disposed within a housing of the vision screening device, the emitter board may include the radiation source and configured to emit the radiation through the beam splitter and out of the housing. The emitter board may be a first emitter board and the vision screening device further may include a second emitter board disposed between the beam splitter and an emission surface of the vision screening device, the second emitter board may include a second radiation source and defining an opening configured to enable the radiation to emit outwards and to enable reflected radiation to pass through and reach the beam splitter. The emitter board may include the white light source and the radiation source.

In an illustrative example, one general aspect includes a vision screening device having a housing with a patient-facing side and a user-facing side. The vision screening device includes a radiation source configured to emit radiation of a first wavelength out of an emission surface of the patient-facing side, a white light source configured to emit white light out of the emission surface, a sensor configured to capture radiation reflected by an eye of a patient and further configured to capture a color image of the eye of the patient, an autofocus system to enable determination of distance data representing a distance to a patient, a first display disposed on the patient-facing side and configured to output characters for one or more vision screening tests, and a second display disposed on the user-facing side and configured to output distance data and output data representative of results of the one or more vision screening tests.

Implementations may include one or more of the following features. The radiation source may include a near-infrared (NIR) band of electromagnetic spectrum. The vision screening device may include a beam splitter positioned between the radiation source and the emission surface, the beam splitter positioned to enable the radiation from the radiation source to exit the housing at the emission surface along a first direction and to reflect received radiation reflected from the patient along a second direction opposite the first direction towards the sensor. An optical axis of the sensor is perpendicular to an emission axis of the radiation source and the white light source, the emission axis extending outwards from the emission surface to the patient. The vision screening device may include an emitter board including the radiation source and the white light source. The emitter board is disposed within the housing with the beam splitter positioned between the emitter board and the emission surface. The vision screening device may include a second emitter board disposed between the beam splitter and the emission surface, the second emitter board may include a second radiation source and defining an opening configured to enable the radiation to emit outwards and to enable reflected radiation to pass through and reach the beam splitter. The vision screening device may include a polarizing filter positioned at or adjacent a patient-facing side of the device.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates an example vision screening device showing a clinician-facing display and interface, in accordance with one or more examples.

FIG. 2 illustrates a first perspective view of an example vision screening device, in accordance with one or more examples.

FIG. 3 illustrates a second perspective view of the example vision screening device of FIG. 2, in accordance with one or more examples.

FIG. 4 illustrates a perspective view of an example vision screening device with a patient facing cover removed, in accordance with one or more examples.

FIG. 5 illustrates a section view of an example vision screening device, in accordance with one or more examples.

FIG. 6 illustrates a section view of an example vision screening device, in accordance with one or more examples.

FIG. 7 illustrates a system architecture of a vision screening device, in accordance with one or more examples.

FIG. 8 illustrates a system architecture of a vision screening system for performing vision screening tests and recording test result data for patients, in accordance with one or more examples.

FIG. 9 illustrates a method for operating the vision screening device, in accordance with one or more examples.

### DETAILED DESCRIPTION

The present disclosure is directed to, in part, a vision screening device, and corresponding methods. Such an example vision screening device may be configured to perform one or more vision screening tests on a patient and to output the results of the vision screening test(s) to an operator of the device, such as a clinician or a physician's assistant. Specifically, the present disclosure is directed to devices and methods for conducting a plurality of screening tests for diseases and abnormalities of the eye. For example, the vision screening device may capture one or more images of the eye illuminated by radiation of different wavelength ranges of electromagnetic spectrum (e.g., infrared, near-infrared, and visible light). The device may determine, based on analysis of the captured images, one or more diseases and/or abnormalities of the eyes, such as cataracts, tumors, ametropia, foreign body in the eye, corneal abrasions, retinal detachment or lesions, congenital conditions and the like, associated with one or both eyes of the patient. Furthermore, the vision screening device may conduct each of the screening tests from a common distance as to remove the requirement of moving the patient or recalibrating the machine.

Based at least in part on the on analysis of the captured images, the device may generate an output including at least one of a recommendation or a diagnosis associated with the patient. Such an output (e.g., the recommendation and/or the diagnosis) may be indicative of diseases or abnormalities detected, indication that the patient requires additional screening, or an indication that the screening was normal (e.g., did not indicate any diseases or abnormalities). For example, the device may determine differences between an image of the left eye and an image of the right eye of the patient, and compare the differences to standard testing data corresponding to normal eyes to provide the recommendation and/or diagnosis.

In some instances, a suite or plurality of tests may be ran using a variety of techniques (visible light, infrared light, etc.) during analysis of a patient's eyes. Furthermore, the device may be configured to conduct each of the tests at a common distance without recalibrating or moving the device and/or patient. In particular, the standard testing data may provide one or more thresholds or a range of values, and the output generated by the device may be based on the differences being less than the threshold(s) or being within the range of values. The device may also generate visualizations of the captured images for displaying to the clinician or the operator of the vision screening device to assist the clinician or the operator in determining a diagnosis. As such, the methods described herein may provide an automated diagnosis based on the analysis of images captured by the vision screening device. The methods described herein may also provide an automated recommendation based on and/or indicative of such a diagnosis.

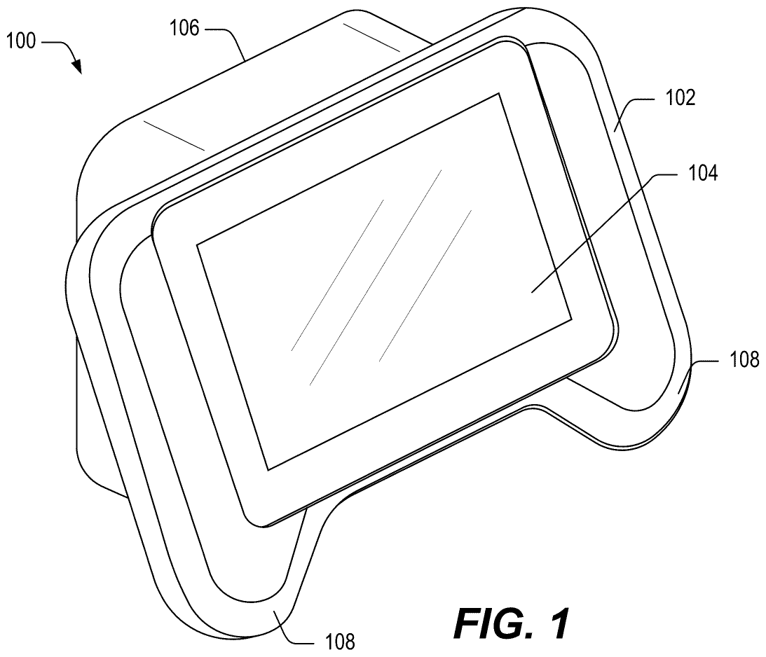

Now in reference to the figures, FIG. 1 depicts an exemplary vision screening device associated with screening for diseases and abnormalities of the eyes. In particular, the screening device may include components for capturing images of the eye(s) of the patient under near-infrared as well as visible light radiation and conducting other ocular assessments such as mobility, color recognition, and/or visual acuity.

The device may include components for controlling the emission of both near-infrared and visible light radiation and the corresponding components configured for capturing the reflected radiation from the eye(s) during the screening. In examples, near-infrared images may be captured before initiating the capture of visible light images, so that pupils of the eyes(s) of the patient do not constrict, or accommodate, during the screening in response to visible light, and the screening may be completed without the need for dilation of the eyes. The device may further include components for analyzing the captured images to determine disease conditions and/or abnormalities in the eye(s) of the patient, and components for determining and reporting of output(s) indicating the disease conditions and/or abnormalities detected during the screening.

Furthermore, the device may incorporate components, such as an output device, which may be configured to display images or graphics associated with visual acuity, mobility, and/or color recognition tests.

In some instances, each of the components described above for conducting various screening tests may be configured or calibrated to execute the various screening tests at a uniform or consistent distance. In other words, each of the screening tests may be conducted by the exemplary vision screening device without changing the position of the device or the patient and without recalibrating the components described above.

The vision screening device may include an eccentric photorefraction exam that is used to evaluate a patient for gaze angle deviations and perform external eye evaluations (e.g., related to the eyelid, sclera, conjunctiva, etc.). The vision screening device may also be used to perform ocular mobility tests that evaluate gaze angles and tracking. The vision screening device may perform color vision evaluations, visual acuity exams, and red reflex tests, among other evaluations.

Additional details pertaining to the above-mentioned devices and techniques are described below with reference to FIGS. 1-9. It is to be appreciated that while these figures describe devices and systems that may utilize the claimed methods, the methods, processes, functions, operations, and/or techniques described herein may apply equally to other devices, systems, and the like.

FIG. 1 illustrates a vision screening device 100 showing a clinician-facing display and interface, in accordance with one or more examples. The vision screening device 100 is illustrated with a housing 102 that encloses the components described below. The housing 102 is depicted with protrusions 108 that may enable a user to grasp the edges of the vision screening device 100 during use.

As illustrated in FIG. 1, in some examples an operator may administer vision screening tests, via a vision screening device 100, on a patient to determine eye health of the patient. As described herein, the vision screening device 100 may perform one or more vision screening tests, including screening for diseases and/or abnormalities of eye(s) when the eyes are illuminated by visible light. In addition, the vision screening device 100 may also be configured to perform other vision screening tests, such as a visual acuity test, a refractive error test, an accommodation test, dynamic eye tracking tests, color vision screening test and/or any other vision screening tests, configured to evaluate and/or diagnose the vision health of the patient. In examples, the vision screening device 100 may comprise a portable device configured to perform the one or more vision screening tests. Due to its portable nature, the vision screening device 100 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screen device 100 being capable of being used for large group testing. It is also envisioned that the vision screening device 100 may be used for administering vision screening tests to all age groups, including newborns and young children and geriatric patients.

As described herein, the vision screening device 100 may be configured to perform one or more vision screening tests on the patient. In examples, one or more vision screening tests may include illuminating the eye(s) of the patient with infrared or near-infrared (NIR) radiation and capturing reflected radiation from the eye(s) of the patient. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient with visible light, and capturing color image(s) of the eye(s) under visible light illumination. The vision screening device 100 may acquire data comprising color images and/or video data of the eye(s) under visible light illumination, and detect pupils, retinas, and/or lenses of the eye(s) of the patient. This data may be used to determine differences between left and right eyes, compare the captured images with standard images, or generate visualizations to assist the operator or a clinician in diagnosing diseases and abnormalities of the eye(s) of the patient. The vision screening device 100 may transmit the data to a vision screening system for analysis to determine an output associated with the patient. Alternatively, or in addition, the vision screening device 100 may perform some or all of the analysis locally to determine the output.

Indeed, in any of the examples described herein, some or all of the disclosed methods may be performed in whole or in part by the vision screening device 100 independently (e.g., without the vision screening system or its components), or by the vision screening system independently (e.g., without the vision screening device 100 or its components). For instance, in some examples, the vision screening device 100 may be configured to perform any of the vision screening tests, and/or other methods described herein without being connected to, or otherwise in communication with, the vision screening system. In some examples, the vision screening system may include one or more components that are similar to and/or the same as those included in the vision screening device 100, and thus, the vision screening system may be configured to perform any of the vision screening tests, and/or other methods described herein without being connected to, or otherwise in communication with, the vision screening device 100.

The vision screening device 100 may include one or more radiation source(s) (not shown in FIG. 1) configured to perform functions associated with administering one or more vision screening tests. The radiation source(s) may comprise individual radiation emitters, such as light-emitting diodes (LEDs), which may be arranged in a pattern to form an LED array. In examples, the radiation source(s) may include near-infrared (NIR) radiation emitters, such as NIR LEDs, for measuring the refractive error of the eye(s) of the patient using photorefraction methods. The NIR radiation emitters of the radiation source(s) may also be used for measuring the gaze angle or gaze direction of the eye(s) of the patient. In addition, the radiation source(s) may also include color LEDs for generating color stimuli for display to the patient during a color vision screening test.

The vision screening device 100 may also include one or more radiation sensor(s), such as infrared cameras, configured to capture reflected radiation from the eye(s) of the patient during the vision screening test(s). For example, the vision screening device 100 may emit, via the radiation source(s), one or more beams of radiation, and may be configured to direct such beams at the eye(s) of the patient. The vision screening device 100 may then capture, via the radiation sensor(s), corresponding radiation that is reflected back (e.g., from pupils of the eye(s)). In examples, the radiation sensor(s) may comprise NIR radiation sensor(s) to capture reflected NIR radiation while the eye(s) of the patient are illuminated by the NIR radiation source(s). The data captured by the NIR radiation sensor(s) may be used in the measurement of the refractive error and/or gaze angle(s) of the eye(s) of the patient. The data may include images and/or video of the pupils, retinas, and/or lenses of the eyes of the patient. In some examples, the images and/or video may be in grayscale (e.g., with values between 0 and 128, or between 0 and 256). The data may be captured intermittently, during specific periods of the vision screening test(s), or during the entire duration of the test(s). Additionally, the vision screening device 100 may process the image(s) and/or video data to determine change(s) in the refractive error and/or gaze angle(s) of the eye(s) of the patient. The grayscale images of the eye(s) captured under NIR illumination may also be used for screening for diseases and abnormalities of the eye(s) such as ametropia, strabismus, and occlusions.

In examples, the vision screening device 100 may further include visible white light source(s) and a camera configured to capture color images and/or video of the eyes under illumination by the white light source(s). The white light source(s) may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) may also be configured to produce white light of different intensity levels. The camera may be configured to capture white light reflected from the eyes of the patient to produce digital color images and/or video. In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 100 may also include one or more display screen(s), such as display 104, which may be color LCD (liquid crystal display), or OLED (organic light-emitting diode) display screens. The display 104 may be an operator display screen facing a direction towards the operator, configured to provide information related to the vision screening tests to the operator. In any of the examples described herein, the display 104 facing the operator may be configured to display and/or otherwise provide the output generated by the vision screening device 100 and/or generated by the vision screening system. The output may include testing parameters, current status and progress of the screening test(s), measurements(s) determined during the test(s), image(s) captured or generated during the screening test(s), a diagnosis determined based on one or more tests, and/or a recommendation associated with the diagnosis. The display 104 facing the operator may also display information related to or unique to the patient, and the patient's medical history.

In some examples, the vision screening device 100 may also include a display screen (not shown in FIG. 1) facing in a direction towards the patient and configured to display content to the patient. The content may include attention-attracting images and/or video to attract attention of the patient and hold the patient's gaze towards the vision screening device 100. Content corresponding to various vision screening test(s) may also be presented to the patient on the display screen. For example, the display screen may display color stimuli to the patient during a color vision screening test, or a Snellen eye chart during a visual acuity screening test. The display screens may be integrated with the vision screening device 100, or may be external to the device, and under computer program control of the vision screening device 100.

The vision screening device 100 may include additional sensors within the housing 102 including a distance sensor such as a range finder, an ambient light sensor, ambient infrared sensor, and other such sensors and components as described herein and as may be used by a clinician during a vision screening exam.

In some examples, the vision screening device 100 may include an autofocus system, the autofocus system may use sensors to detect a focal point and adjust the imaging components of the vision screening device 100 to increase or reach a maximum sharpness value. In some examples, the vision screening device 100 may identify areas of high contrast where distinct differences between adjacent pixels may be more obvious. The lens or other imaging components may adjust to change the plane of focus until the area of focus achieves maximum contrast. In some examples, the autofocus system may split incoming light into two separate images and send them to a dedicated autofocus sensor. The vision screening device 100 and specifically a computing component may compare the two images, and by comparing or measuring the displacement the vision screening device 100 may determine how far to move the lens or other imaging components to achieve focus. The autofocus system may also be used to determine or estimate the distance to the patient.

Depicted in FIG. 1, the vision screening device 100 includes a display 104 that may be used by a clinician or other user to interact with the vision screening device 100. The display 104 may be used for inputting information related to a patient, selecting test parameters, adding additional evaluation inputs, and otherwise controlling the vision screening device 100. As depicted, the display 104 may be a touch-screen or other similar display that enables user input through the display 104 while also providing output to the clinician.

On a side of the housing 102 opposite from the display 104 is a patient-facing surface 106. The patient-facing surface 106 includes an emission surface through which light and other radiation sources are emitted towards a patient during an exam. Additionally, the patient-facing surface 106 enables a distance sensor enclosed within the housing (not shown in FIG. 1) to gather distance data for use in positioning the vision screening device 100 relative to a patient for evaluation.

The vision screening device 100 may be used for various evaluations at prescribed distances for distance vision testing. For example, vision testing and evaluation may be performed at a distance of five feet from the patient. The distance between the patient and the vision screening device is important for photorefraction exams (typically performed at a distance of three feet in previous systems) as well as visual acuity tests (typically tested at distances between ten and twenty feet). The vision screening device 100 is designed to perform the various tests and evaluation at a common distance for ease and speed of testing. The single common distance enables simpler exam processes as patients need not be shifted for different tests. Additional vision tests such as color vision and near vision may also be performed at the common distance (e.g., five feet or ten feet). Further still, to aid in vision screening, the vision screening device may be programmed to perform a sequence of exams simultaneously or back-to-back without requiring a reset or changing parameters of the vision screening device 100. In some examples, the clinician may use the vision screening device 100 to perform a suite of tests, or may select a subset of tests to perform during an evaluation.

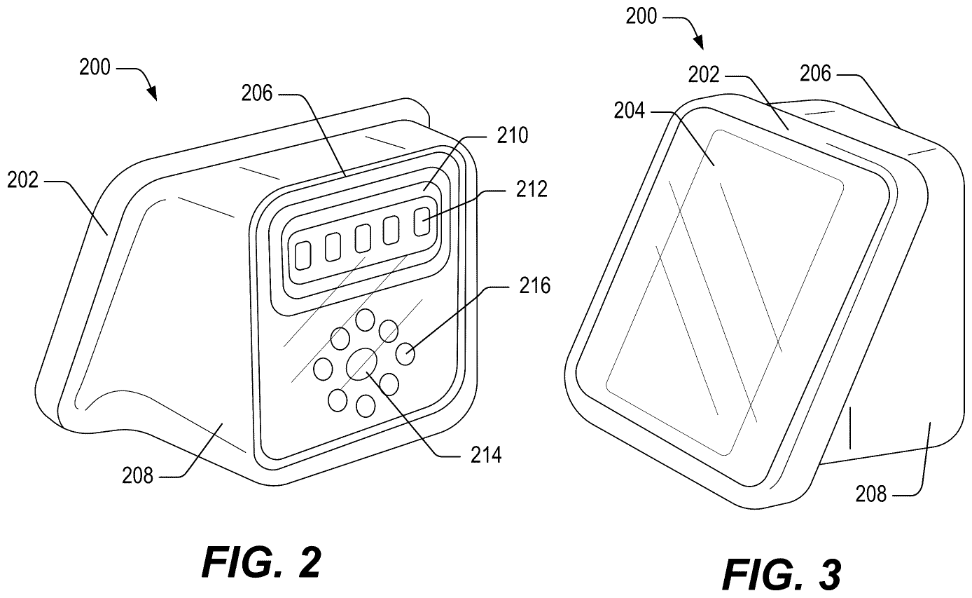

FIGS. 2-3 illustrate perspective views of an example vision screening device 200, in accordance with one or more examples. The example vision screening device 200 includes a housing 202 similar or identical to the housing 102. Additionally, the vision screening device 200 includes a display 204 similar or identical to the display 104 and an emission surface 206 on a patient-facing side. The emission surface 206 provides a surface through which light and/or other emissions may be projected towards a patient and also enables reflected light and other signals to be received. The housing 202 encloses a space 208 that receives reflected light and NIR light after it reflects off the patient. The emission surface 206 may be transparent to such light and signal transmissions, or include transparent portions, for example around a second display 210 and emitters 214 and aperture 216.

The second display 210 faces the patient and is used to display digital objects 212 such as letters, images, shapes, and other such digital display objects. The second display 210 may display the various digital objects 212 at different sizes or heights, for example to test visual acuity based on digital objects 212 of decreasing height. The second display 210 transmits digital information outwards towards the patient for the patient to interact with (e.g., read) while the emitters 214 and aperture 216 are used to gather information regarding the eyes of the patient.

The emitters 214 and aperture 216 are used to emit NIR light and white light towards the patient and receive reflected light off the patient at the aperture 216. The reflected light passes through the aperture 216 and into the interior of the housing 202 where it is reflected to a sensor (e.g., camera) or multiple cameras that may be used to detect the white light and NIR reflection data.

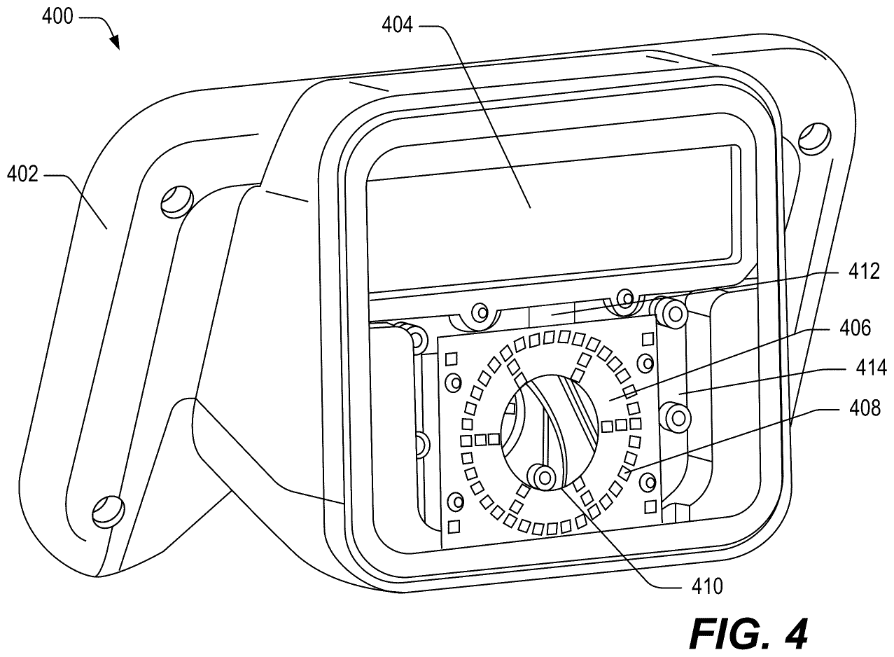

FIG. 4 illustrates a perspective view of an example vision screening device 400 with a patient facing cover removed, showing a partial view of internal components stored within a housing 402 of the vision screening device 400, in accordance with one or more examples. In addition to the housing 402, the vision screening device 400 includes a display 404 which serves as a patient-facing display to display digital objects such as symbols, pictures, letters, and other such information to a patient during an exam.

With the cover removed, an interior of the housing 402 is depicted including an emitting board 406 that includes emitters 408. The emitting board 406 provides power from a power source of the vision screening device 400 to the emitters 408 and also provides for a processor of the vision screening device 400 to control the emitters 408 to selectively emit radiation towards the patient. The emitters 408 may include NIR emitters as well as visible light emitters (e.g., white light emitters) that project light out of the patient-facing side of the vision screening device 400 towards the patient. The emitters 408 may include LEDs and other sources of near infrared light as well as visible light or other types of emitted radiation that may be directed towards the patient and received back at the vision screening device 400 after reflecting off the patient.

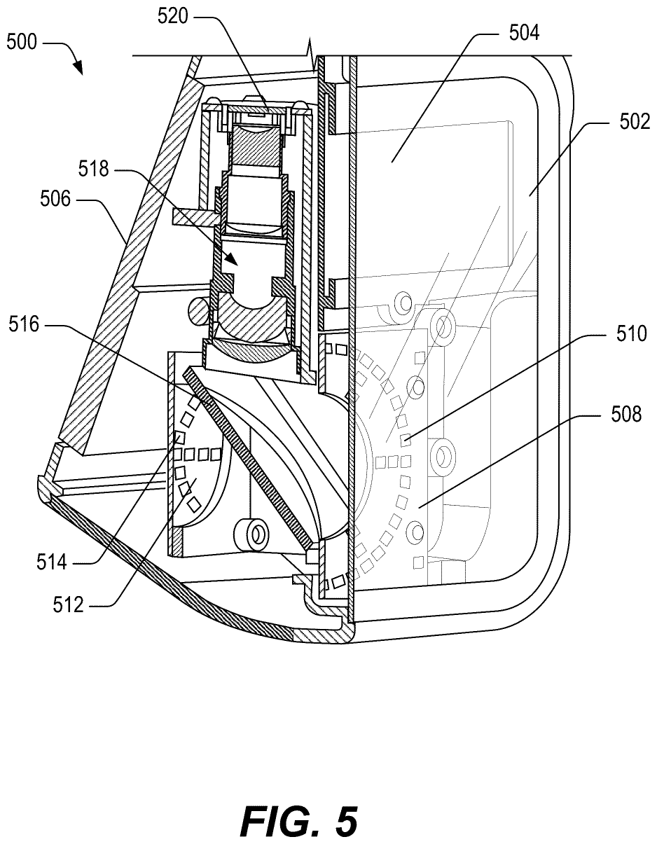

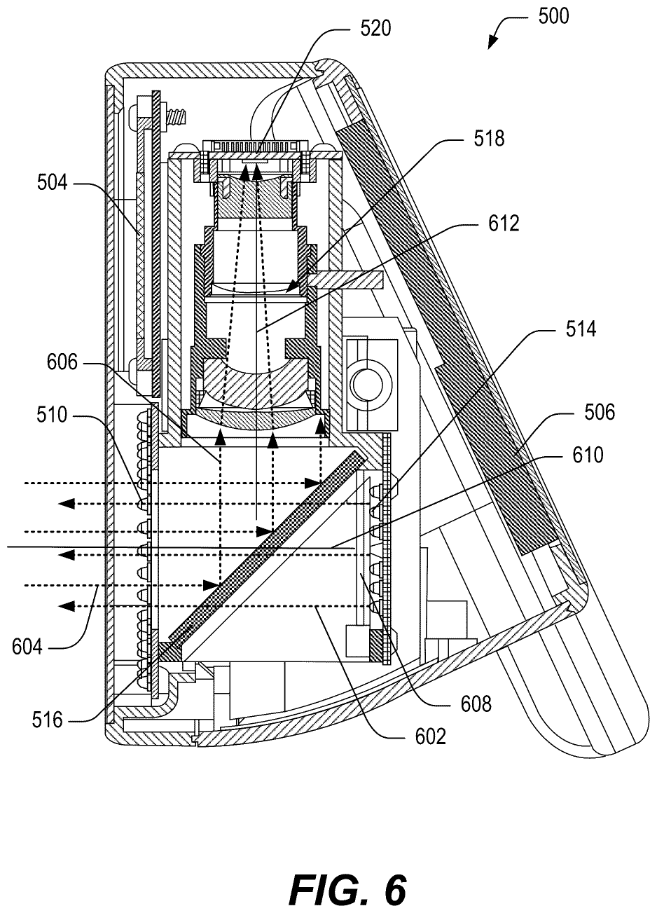

The emitting board 406 defines an opening 410 at a center of the array of the emitters 408. The array of emitters 408 may share an optical axis and may direct light or radiate energy outwards toward the patient along near parallel directions. The reflected energy from the patient passes through the opening 410 to reach further internal components of the vision screening device 400 for sensing and detection, as depicted in FIGS. 5-6.

The vision screening device 400 further includes a sensor 412. In FIG. 4 the sensor 412 is depicted positioned facing the patient and disposed vertically underneath the display 404. In some examples, the sensor 412 may be positioned within the housing 402 and any suitable position facing the patient-side of the housing 402 and oriented to face the patient when in use. The sensor 412 may include an ultrasonic sensor (and be positioned at the rear surface of the housing 402 with no glass between the sensor 412 and the patient, a millimeter wave radar sensor, a light-base sensor, or other such distance measuring sensor.

The sensor 412 may include an autofocus sensor to detect a focal point and adjust the imaging components of the vision screening device 400 to increase or reach a maximum sharpness value. In some examples, the vision screening device 400 may identify areas of high contrast where distinct differences between adjacent pixels may be more obvious. The lens or other imaging components may adjust to change the plane of focus until the area of focus achieves maximum contrast. In some examples, the autofocus system may split incoming light into two separate images and send them to a dedicated autofocus sensor. The vision screening device 400 and specifically a computing component may compare the two images, and by comparing or measuring the displacement the vision screening device 400 may determine how far to move the lens or other imaging components to achieve focus. The autofocus system may also be used to determine or estimate the distance to the patient.

The vision screening device 400 is shown with a rigid chassis 414 that floats within the housing 402. The optical components of the vision screening device including the emitters, beam splitters, lenses, and filters are aligned and calibrated and secured to the rigid chassis 414. In examples, the emitters of the LED boards, lenses, image sensors are all aligned and centered for capturing image data. The rigid chassis 414 floats within the housing, and may be mounted to the housing through one or more shock absorbers, energy absorbing devices, and other such components to provide protection for the components in the event the vision screening device 400 is dropped. In examples, one or more of the components, such as the clinician-facing display and/or patient-facing display may be surface mounted to a skin of the housing 402. The housing 402 further includes one or more access ports for servicing the interior components, for example to calibrate or re-align the optical components.

FIGS. 5-6 illustrate a section view of an example vision screening device 500, in accordance with one or more examples. In the section view, the vision screening device 500 is shown with elements of the vision screening device shown and described with respect to FIGS. 1-4 above, including a housing 502, display 504, display 506, emitting board 508, and emitters 510. The section view of FIG. 5 illustrates aspects of the interior of the vision screening device including a second emitting board 512, emitters 514, beam splitter 516, lens and filter assembly 518, and sensor 520.

Within the vision screening device 500, and positioned behind the emitting board 508 is a beam splitter 516 that is used to split light based on the direction from which the light is incident. Light reaching the beam splitter 516 from within the housing (e.g., behind the beam splitter 516) that is emitted by the emitters 514 of the second emitting board 512, is transmitted through a polarizer (not shown in FIG. 5) and then through the beam splitter and remains traveling in a direction parallel or substantially parallel with the direction or incident angle of the incoming light. The polarizer may be positioned between the beam splitter and the emitter board 512. In contrast, reflected light that reaches the beam splitter 516 from the environment (and passes through an emitting surface of the vision screening device 500) is reflected by the beam splitter towards the lens and filter assembly 518 and finally to the sensor 520 where the reflected light is received and subsequently detected and processed as described herein. In some examples, the beam splitter 516 may be a plate beam splitter with a coating that causes the incident light originating from outside the vision screening device 500 to be reflected upwards (as oriented in FIG. 5) and into the lens and filter assembly 518. In examples, the beam splitter 516 may be accompanied with one or more additional filters or elements in the light path of the emitted NIR and/or visible light. The filter may, for example be a polarizing filter that polarizes the light as it is emitted from the vision screening device (e.g., after passing through the beam splitter from the emitters 514) and when the reflected light is returned, the beam splitter 516 may reflect the polarized light into the lens and filter assembly 518.

The lens and filter assembly 518 may include various filters including polarizing filters, filters to remove particular types of light, notch filters (e.g., to remove a portion of red light or other such light ranges), or otherwise treat the visible light and/or NIR light as it travels to the sensor 520. In examples, the filters may include coatings disposed on one or more lenses of the lens and filter assembly 518. The sensor 520 may include one or more sensors, such as a first sensor that detects NIR electromagnetic radiation and a second sensor that detects visible light. In examples, the sensor 520 may be a single sensor equipped to detect both NIR electromagnetic radiation as well as visible light. The light received at the sensor 520 may be processed based on the particular type of exam being performed either by an on-board processor and/or an external processing system to provide an output that may be displayed at the display 506 and/or output to an external system such as a system that hosts an electronic medical record for the patient.

FIG. 6 illustrates a section view of the vision screening device 500 and is illustrated including light traces of emitted radiation and received reflected radiation. Emitted light 602 includes light from emitters 514 that extends along a first optical axis 610 including along a direction parallel to the first optical axis 610 and may include visible night, NIR, white light, blue light, red light, green light, or other such light beams as emitted from the emitters 514. The emitted light 602 passes from the emitters 514 through a polarizer 608 and then through the beam splitter 516. The emitted light then passes out of the housing 502. Additional emitted light emanates from emitters 510 and joins the emitted light 602 traveling parallel with the first optical axis 610. After reflecting off an environment, including the patient, reflected light 604 is returned to the housing 502. The reflected light 604 may come in through an aperture in the housing 502 and may be parallel to the first optical axis but in a direction opposite the emitted light 602 then reaches the beam splitter 516 and some or all of the reflected light 604 is reflected to a second optical axis 612 perpendicular to the first optical axis 610 as diverted light 606. Examples, the diverted light 606 may include a portion of the reflected light 604. In examples the portion may be in a range of ten percent to one hundred percent. The second optical axis 612 enables the system to receive and process the diverted light 606 at the sensor 520 and provides for a greater distance over which the reflected light 604 and diverted light 606 may be conditioned through lenses and filters before reaching the sensor 520. The additional distance is enabled within a housing that maintains a compact handheld footprint that remains thing and similar in shape and form factor to a tablet.

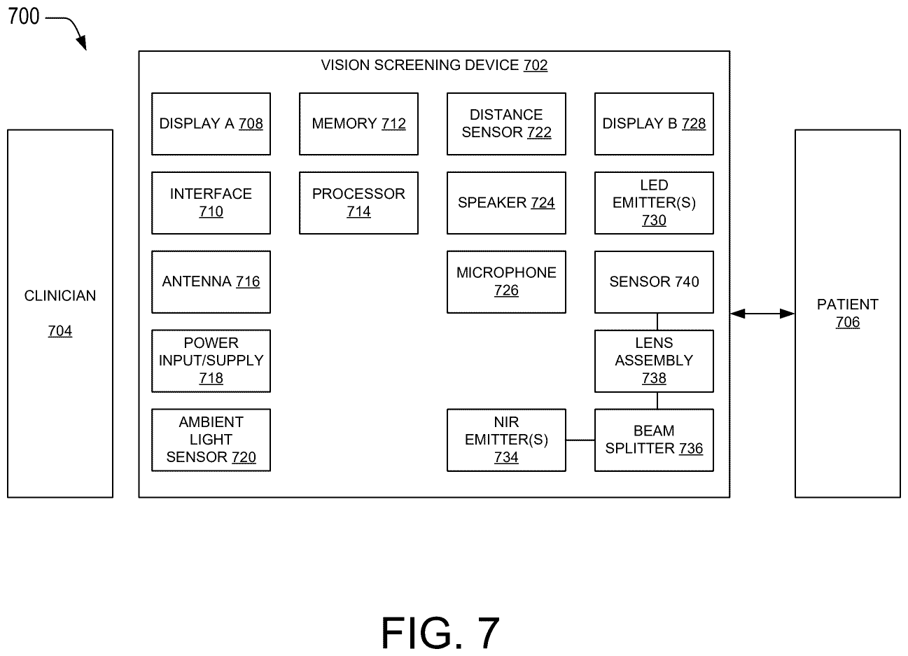

FIG. 7 illustrates a system architecture 700 of a vision screening device 702, in accordance with one or more examples. The vision screening device 702 may be used by a clinician 704 interacting with a patient 706, as described herein. The system architecture 700 illustrates a subset of components that may be included as part of the vision screening device 702 in an illustrative example. The vision screening device 702 may include additional components not shown herein that may be used to perform the operations described herein and/or support one or more additional components. Further, other components may be substituted for one or more of the shown components that may perform the same or similar functions.

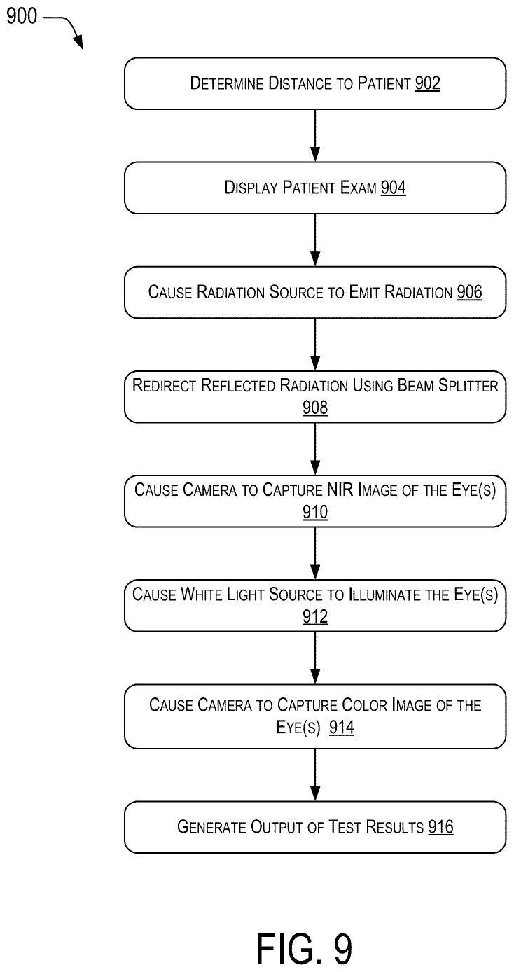

The vision screening device 702 includes a display A 708 that is clinician-facing and provides an interface for a clinician to input data as well as receive outputs from the vision screening device. A further interface 710, which may include a touchscreen display, provides the clinician with an ability to interact with the vision screening device, for example to input demographic data, patient data, select types of exams to perform, and other such interactions. A memory 712, such as a non-transitory computer-readable medium may include specific instructions (e.g., software) that, when executed by a processor 714, cause the processor to perform various operations, such as the operations of FIG. 9, or other operations described herein.

The vision screening device 702 further includes an antenna 716 that may provide a wireless communication with one or more other devices or systems such as a system that stores or provides access to electronic medical records for patients. A power input/supply 718 provides for portable power (e.g., batteries) as well as a system for charging batteries and/or providing consistent power to one or more other components of the vision screening device 702.

An ambient light sensor 720 may be used to detect light levels and/or characteristics of ambient light in the environment surrounding the visions screening device. The ambient light sensor 720 may detect a brightness of the environment and may also detect illumination colors, for instance if the lighting in a particular area is “warm” or “cool” relating to the temperature of the lighting. In some examples, the ambient light sensor 720 may be used to detect potential environmental light that may interfere with the vision screening tests, such as a prevalence of NIR light from a nearby source. Such information may be displayed on display A 708 for the clinician to either acknowledge or work to adjust environmental conditions to be more conducive to particular eye exams.

A sensor 722 may be used to determine a distance between the vision screening device 702 and the patient 706. The sensor 722 may include an autofocus sensor, distance sensor, or range finding sensor of any suitable type for detecting a distance between the location of the sensor (at the vision screening device 702) and the patient 706. In examples, the sensor 722 may be positioned behind a patient-facing cover on a side of the vision screening device 702 opposite display A 708. Accordingly, as with the imaging components, the sensor 722 may enable the housing of the vision screening device 702 to remain compact and user-friendly by presenting a slim and easily held device that is also portable.

A speaker 724 may provide audible outputs or cues, such as a beeping sound indicative of the patient being at or near the predetermined distance. In an example, an audible cue may increase in tone and/or frequency of a repeated sound as the patient 706 approaches the predetermined distance. A microphone 726 may be used to gather audible data, for example to enable the vision screening device 702 to recognize the patient reading a particular chart and to provide evaluation of their performance reading the symbols displayed on display B 728, that is user-facing, as described herein.

LED emitter(s) 730 may include NIR emitters 734 as well as white light emitters that provide light to reflect off the patient 706, with the reflected radiation received through a beam splitter 736 and lens assembly 738 before arriving at the sensor 740, as shown and described with respect to FIGS. 5-6 herein.

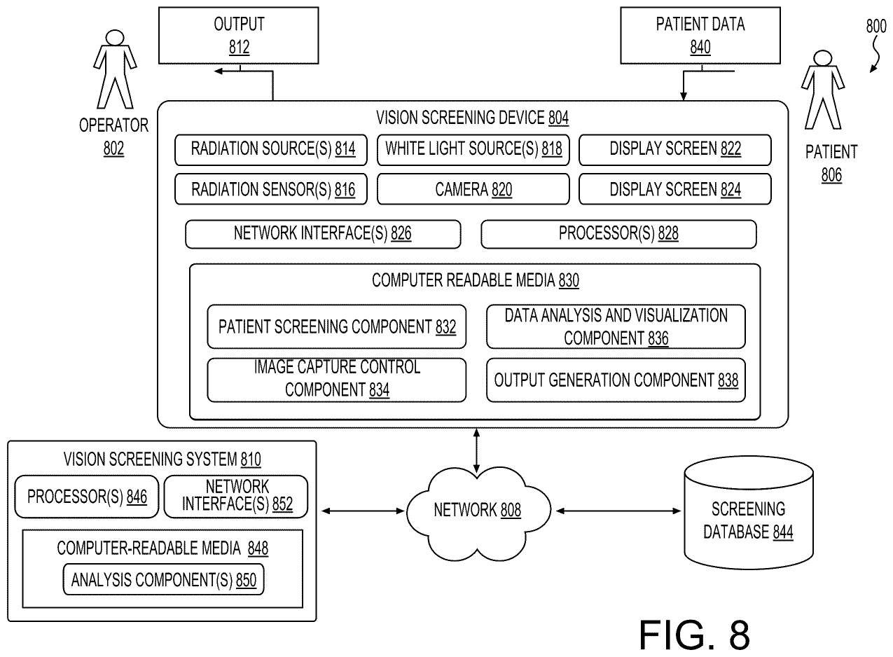

FIG. 8 illustrates a system architecture of a vision screening system for performing vision screening tests and recording test result data for patients, in accordance with one or more examples. As illustrated in FIG. 8, in some examples an operator 802 may administer vision screening tests, via a vision screening device 804, on a patient 806 to determine eye health of the patient 806. As described herein, the vision screening device 804 may perform one or more vision screening tests, including screening for diseases and/or abnormalities of eye(s) when the eyes are illuminated by visible light. In addition, the vision screening device 804 may also be configured to perform other vision screening tests, such as a visual acuity test, a refractive error test, an accommodation test, dynamic eye tracking tests, color vision screening test, visible light test, and/or any other vision screening tests, configured to evaluate and/or diagnose the vision health of the patient 806. In examples, the vision screening device 804 may comprise a portable device configured to perform the one or more vision screening tests. Due to its portable nature, the vision screening device 804 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screening device 804 being capable of being used for large group testing. It is also envisioned that the vision screening device 804 may be used for administering vision screening tests to all age groups, including newborns and young children and geriatric patients.

As described herein, the vision screening device 804 may be configured to perform one or more vision screening tests on the patient 806. In examples, one or more vision screening tests may include illuminating the eye(s) of the patient 806 with infrared or near-infrared (NIR) radiation and capturing reflected radiation from the eye(s) of the patient 806. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient 806 with visible light, and capturing color image(s) of the eye(s) under visible light illumination. The vision screening device 804 may acquire data comprising color images and/or video data of the eye(s) under visible light illumination, and detect pupils, retinas, and/or lenses of the eye(s) of the patient 806. This data may be used to determine differences between left and right eyes, compare the captured images with standard images, or generate visualizations to assist the operator 802 or a clinician in diagnosing diseases and abnormalities of the eye(s) of the patient. The vision screening device 804 may transmit the data, via a network 808, to a vision screening system 810 for analysis to determine an output 812 associated with the patient 806. Alternatively, or in addition, the vision screening device 804 may perform some or all of the analysis locally to determine the output 812. Indeed, in any of the examples described herein, some or all of the disclosed methods may be performed in whole or in part by the vision screening device 804 independently (e.g., without the vision screening system 810 or its components), or by the vision screening system 810 independently (e.g., without the vision screening device 804 or its components). For instance, in some examples, the vision screening device 804 may be configured to perform any of the vision screening tests, and/or other methods described herein without being connected to, or otherwise in communication with, the vision screening system 810 via the network 808. In other example, the vision screening system 810 may include one or more components that are similar to and/or the same as those included in the vision screening device 804, and thus, the vision screening system 810 may be configured to perform any of the vision screening tests, and/or other methods described herein without being connected to, or otherwise in communication with, the vision screening device 804.

As shown schematically in FIG. 8, the vision screening device 804 may include one or more radiation source(s) 814 configured to perform functions associated with administering one or more vision screening tests. The radiation source(s) 814 may comprise individual radiation emitters, such as light-emitting diodes (LEDs), which may be arranged in a pattern to form an LED array. In examples, the radiation source(s) 814 may include near-infrared (NIR) radiation emitters, such as NIR LEDs, for measuring the refractive error of the eye(s) of the patient 806 using photorefraction methods. The NIR radiation emitters of the radiation source(s) 814 may also be used for measuring the gaze angle or gaze direction of the eye(s) of the patient 806. In addition, the radiation source(s) 814 may also include color LEDs for generating color stimuli for display to the patient 806 during a color vision screening test.

The vision screening device 804 may also include one or more radiation sensor(s) 816, such as infrared cameras, configured to capture reflected radiation from the eye(s) of the patient during the vision screening test(s). For example, the vision screening device 804 may emit, via the radiation source(s) 814, one or more beams of radiation, and may be configured to direct such beams at the eye(s) of the patient 806. The vision screening device 804 may then capture, via the radiation sensor(s) 816, corresponding radiation that is reflected back (e.g., from pupils of the eye(s)). In examples, the radiation sensor(s) 816 may comprise NIR radiation sensor(s) to capture reflected NIR radiation while the eye(s) of the patient 806 are illuminated by the radiation source(s) 814. The data captured by the radiation sensor(s) 816 may be used in the measurement of the refractive error and/or gaze angle(s) of the eye(s) of the patient 806. The data may include images and/or video of the pupils, retinas, and/or lenses of the eyes of the patient 806. In some examples, the images and/or video may be in grayscale (e.g., with values between 0 and 828, or between 0 and 256). The data may be captured intermittently, during specific periods of the vision screening test(s), or during the entire duration of the test(s). Additionally, the vision screening device 804 may process the image(s) and/or video data to determine change(s) in the refractive error and/or gaze angle(s) of the eye(s) of the patient 806. The grayscale images of the eye(s) captured under NIR illumination may also be used for screening for diseases and abnormalities of the eye(s) such as ametropia, strabismus, and occlusions. As described herein, in some examples, the radiation sensor(s) 816 may be combined with camera 820 into a single sensing component that detects both visible light and NIR.

In examples, the vision screening device 804 may further include visible white light source(s) 818 and camera 820 configured to capture color images and/or video of the eyes under illumination by the white light source(s) 818. The white light source(s) 818 may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) 818 may also be configured to produce white light of different intensity levels. The camera 820 may be configured to capture white light reflected from the eyes of the patient to produce digital color images and/or video. The camera 820 may comprise a high-resolution, auto-focus digital camera with custom optics for imaging eyes in clinical applications. The color images and/or video captured by the camera 820 may be stored in various formats, such as JPEG, BITMAP, TIFF, etc. (for images) and MP4, MOV, WMV, AVI etc. (for video). In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 804 may also include one or more display screen(s), such as display screen 822 and display screen 824, which may be color LCD (liquid crystal display), or OLED (organic light-emitting diode) display screens. The display screen 822 may be an operator display screen facing a direction towards the operator 802, configured to provide information related to the vision screening tests to the operator 802. In any of the examples described herein, the display screen 822 facing the operator 802 may be configured to display and/or otherwise provide the output 812 generated by the vision screening device 804 and/or generated by the vision screening system 810. The output 812 may include testing parameters, current status and progress of the screening test(s), measurements(s) determined during the test(s), image(s) captured or generated during the screening test(s), a diagnosis determined based on one or more tests, and/or a recommendation associated with the diagnosis. The display screen 822 facing the operator 802 may also display information related to or unique to the patient, and the patient's medical history.

In some examples, the vision screening device 804 may also include a display screen 824 facing in a direction towards the patient 806 and configured to display content to the patient 806. The content may include attention-attracting images and/or video to attract attention of the patient and hold the patient's gaze towards the vision screening device 804. Content corresponding to various vision screening test(s) may also be presented to the patient 806 on the display screen 824. For example, the display screen 824 may display color stimuli to the patient 806 during a color vision screening test, or a Snellen eye chart during a visual acuity screening test. The display screens 822, 824 may be integrated with the vision screening device 804, or may be external to the device, and under computer program control of the vision screening device 804.

The vision screening device 804 may transmit the data captured by the radiation sensor(s) 816 and/or the camera 820, via the network 808, using network interface(s) 826 of the vision screening device 804. In addition, the vision screening device 804 may also similarly transmit other testing data associated with the vision screening test(s) being administered, (e.g., type of test, duration of test, patient identification and the like). The network interface(s) 826 of the vision screening device 804 may be operably connected to one or more processor(s) 828 of the vision screening device 804, and may enable wired and/or wireless communications between the vision screening device 804 and one or more components of the vision screening system 810, as well as with one or more other remote systems and/or other networked devices. For instance, the network interface(s) 826 may include a personal area network component to enable communications over one or more short-range wireless communication channels, and/or a wide area network component to enable communication over a wide area network. In any of the examples described herein, the network interface(s) 826 may enable communication between, for example, the processor(s) 828 of the vision screening device 804, and the vision screening system 810, via the network 808. The network 808 shown in FIG. 8 may be any type of wireless network or other communication network known in the art. Examples of network 808 include the Internet, an intranet, a wide area network (WAN), a local area network (LAN), and a virtual private network (VPN), cellular network connections and connections made using protocols such as 802.11a, b, g, n and/or ac.

The vision screening system 810 may be configured to receive data, from the vision screening device 804 and via the network 808, collected during the administration of the vision screening test(s). In some examples, based at least in part on processing the data, the vision screening system 810 may determine the output 812 associated with the patient 806. For example, the output 812 may include a recommendation and/or diagnosis associated with eye health of the patient 806, based on an analysis of the color image data and/or NIR image data indicative of diseases and/or abnormalities associated with the eye(s) of the patient 806. The vision screening system 810 may communicate the output 812 to the processor(s) 828 of the vision screening device 804 via the network 808. As noted above, in any of the examples described herein one or more such recommendations, diagnoses, or other outputs may be generated, alternatively or additionally, by the vision screening device 804.

As described herein, a processor, such as the processor(s) 828, can be a single processing unit or a number of processing units, and can include single or multiple computing units or multiple processing cores. The processor(s) 828 can be implemented as one or more microprocessors, microcomputers, microcontrollers, digital signal processors, central processing units, state machines, logic circuitries, and/or any devices that manipulate signals based on operational instructions. For example, the processor(s) 828 can be one or more hardware processors and/or logic circuits of any suitable type specifically programmed or configured to execute the algorithms and processes described herein. As shown schematically in FIG. 8, the vision screening device 804 may also include computer-readable media 830 operably connected to the processor(s) 828. The processor(s) 828 can be configured to fetch and execute computer-readable instructions stored in the computer-readable media 830, which can program the processor(s) 828 to perform the functions described herein.

The computer-readable media 830 may include volatile and nonvolatile memory and/or removable and non-removable media implemented in any type of technology for storage of information, such as computer-readable instructions, data structures, program modules, or other data. Such computer-readable media 830 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 830 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 830 can be used to store any number of functional components that are executable by the processor(s) 828. In examples, these functional components comprise instructions or programs that are executable by the processor(s) 828 and that, when executed, specifically configure the one or more processor(s) 828 to perform actions associated with one or more of the vision screening tests used for the detection and diagnosis of diseases and abnormalities of the eye(s). For example, the computer-readable media 830 may store one or more functional components for administering vision screening tests, such as a patient screening component 832, an image capture control component 834, a data analysis and visualization component 836, and/or an output generation component 838, as illustrated in FIG. 8. At least some of the functional components of the vision screening device 804 will be described in detail below.

In examples, the patient screening component 832 may be configured to store and/or access patient data 840 associated with the patient 806. For example, the patient data 840 may include demographic information such as name, age, ethnicity, and the like. When the vision screening device 804 and/or vision screening system 810 initiates a vision screening test, the patient 806 may provide, or the operator 802 may request, from the patient 806 or a guardian of the patient 806 the patient data 840 regarding the patient's demographic information, medical information, preferences, and the like. In such examples, the operator 802 may request the data while the screening is in progress, or before the screening has begun. In some examples, the operator 802 may be provided with predetermined categories associated with the patient 806, such as predetermined age ranges (e.g., newborn to six months, six to twelve months, one to five years old, etc.), and may request the patient data 840 in order to select the appropriate category associated with the patient 806. In other examples, the operator 802 may be provided a free form input associated with the patient data 840. In still further examples, an input element may be provided to the patient 806 directly.

The vision screening device 804 may use a sensor such as an autofocus sensor and/or a distance sensor to detect distance data corresponding to a distance between the vision screening device 804 and the patient 806. The distance may be used to position the vision screening device 804 relative to the patient, for example to reach a particular spacing or distance between the patient 806 and the vision screening device 804 such as described herein. Additional sensors such as ambient light sensors may be used for determining environmental conditions, such as whether environmental lighting conditions are suitable for a particular eye exam to take place.

Alternatively, or in addition, the vision screening device 804 and/or vision screening system 810 may determine and/or detect the patient data 840 during the vision screening test. For example, the vision screening device 804 may include one or more digital cameras, motion sensors, proximity sensors, or other image capture devices configured to collect images and/or video data of the patient 806, and one or more processors of the vision screening device 804 may analyze the data to determine the patient data 840, such as the age category of the patient 806 or a distance of the patient 806 from the screening device. For example, the vision screening device 804 may be equipped with a range finder, such as an ultra-sonic range finder, an infrared range finder, and/or any other proximity sensor that may be able to determine the distance of the patient 806 from the screening device.

Alternatively, or in addition, the vision screening device 804 may be configured to transmit the images/video data to the vision screening system 810, via the network 808, for analysis to determine the patient data 840. Further, the patient screening component 832 may be configured to receive, access, and/or store the patient data 840 associated with the patient 806 and/or additional patients. For example, the patient screening component 832 may store previous patient information associated with the patient 806 and/or other patients. For instance, the patient screening component 832 may store previous screening history of the patient 806, including data from previous screening such as color images, NIR images, and/or video of the eye(s) of the patient 806. The patient screening component 832 may receive the patient data 840 and/or may access such information via the network 808. For example, the patient screening component 832 may access an external database, such as screening database 844, storing data associated with the patient 806 and/or other patients. The screening database 84 may be configured to store the patient data 840 in association with a patient ID. When the operator 802 and/or the patient 806 enters the patient ID, the patient screening component 832 may access or receive the patient data 840 stored in association with the patient ID of the patient 806.

In examples, the patient screening component 832 may be configured to determine the vision screening test(s) to administer to the patient 806 based at least in part on the patient data 840. For example, the patient screening component 832 may utilize the patient data 840 to determine a testing category that the patient 806 belongs to (e.g., a testing category based on age, medical history, etc.). The patient screening component 832 may determine the vision screening test(s) to administer based on the testing category. For example, if the patient data 840 indicates that the patient is a newborn, the selected vision screening test(s) may include screening for congenital conditions of the eye such as congenital cataracts, retinoblastoma, opacities of the cornea, strabismus and the like. In addition, eye abnormalities may be associated with systemic inherited diseases such as Marfan syndrome and Tay-Sachs disease. For example, a screening test for a characteristic red spot in the eye may indicate Tay-Sachs disease. As another example, if the patient data 840 indicates that the patient is above fifty years old, the patient screening component 832 may determine that the vision screening test(s) include screening for onset of cataracts, macular degeneration and other age-related eye diseases.

The patient screening component 832 may also determine vision screening test(s) based on the patient's medical history. For example, the screening database 844 may store, in the patient data 840, medical history associated with previous vision screening tests of the patient 806, including test results, images of the eye(s), measurements, recommendations, and the like. The patient screening component 832 may access the patient data 840 including medical history from the screening database 844 and determine vision screening test(s) to administer to monitor status and changes in previously detected vision health issues. For example, if a progressive eye disease, such as onset of cataracts or macular degeneration, was detected in a previous screening, further screening may be administered to track the development of the disease. As another example, if the patient 806 had surgery for removal of a tumor of the eye(s), the vision screening test(s) may include screening for further tumors or scarring in the eye(s).

In some examples, the computer-readable media 830 may additionally store an image capture control component 834. The image capture control component 834 may be configured to operate the radiation source(s) 814, the radiation sensor(s) 816, the white light source(s) 818, and the camera 820 of the vision screening device 804, so that images of the eye(s) are captured under specific illumination conditions required for each particular vision screening test(s). As discussed, the radiation source(s) 814 may include NIR LEDs for illuminating the eye(s) during capture of grayscale images for measuring the refractive error and/or gaze angle of the eye(s) of the patient 806, and the white light source(s) 818 may include white light LEDs for illuminating the eye(s) during capture of color images of the eye(s) by the camera 820. In examples, the image capture control component 834 may generate commands to operate and control the individual radiation sources, such as LEDs of the NIR LEDs, as well as the LEDs of the white light source 818. Control parameters of the LEDs may include intensity, duration, pattern and cycle time. For example, the commands may selectively activate and deactivate the individual LEDs of the radiation source(s) 814 and white light sources 818 to produce illumination from different angles as needed by the vision screening test(s) indicated by the patient screening component 832. The image capture control component 834 may activate the NIR LEDs of the radiation source(s) 814 used for measuring the refractive error and/or gaze angle of the eye(s) of the patient 806 in synchronization with the capture of images of the eye(s) by the radiation sensors(s) 816 during the performance of a vision screening test. Similarly, the image capture control component 834 may activate the LEDs of the white light source(s) 818 in synchronization with the capture of color images of the eye(s) by the camera 820.

The individual radiation sources, such as LEDs, of the radiation source(s) 814 or the white light source(s) 818 may be controlled by the image capture control component 834 according to control parameters stored in the computer-readable media 830. For instance, control parameters may include intensity, duration, pattern, cycle time, and so forth, of the NIR LEDs of the radiation source(s) 814 and/or the LEDs producing white light of the white light source(s) 818. For example, the image capture control component 834 may use the control parameters to determine a duration that individual LEDs of the radiation source(s) 814, 818 emit radiation (e.g., 50 milliseconds, 800 milliseconds, 200 milliseconds, etc.). Additionally, the image capture control component 834 may utilize the control parameters to alter an intensity and display pattern of NIR LEDs of the radiation source(s) 814 for the determination of refractive error of the eye(s) based on photorefraction and/or gaze angle of the eye(s). With respect to intensity, the image capture control component 834 may control parameters to direct the LEDs of the white light source(s) 818 to emit light at an intensity that is bright enough to capture a color image of the eye(s) using the camera 820, while also limiting brightness to avoid or reduce pupil constriction or accommodation. The image capture control component 834 may also control the intensity of the white light source(s) 818 to gradually increase the intensity at a certain rate while activating the camera 820 to capture images and/or video of the eyes to record response of the pupils of the patient's eyes to the increasing intensity of illumination.

Further, the image capture control component 834 may order the emission of radiation from the radiation source(s) 814, 818 so that the NIR LEDs are activated and the images of the eye(s) under NIR radiation are captured before the activation of the LEDs of the white light source(s) 818. In some examples, this ordering may prevent the constriction of the pupils of the eye(s) in response to white light impinging upon them, and/or may allow for the capture of images of the internal structures of the eye(s) without the need for dilating the pupils of the patient 806. In some examples, the image capture control component 834 may additionally control the radiation source(s) 814, 818 to generate patterns such as circular patterns, alternating light patterns, flashing patterns, patterns of shapes such as circles or rectangles, and the like to attract the attention of the patient 806, and/or control color LEDs of the radiation source(s) 814, 818 to display color stimuli such as color dot patterns to the patient 806 during vision screening.

The image capture control component 834 may also control the radiation sensor(s) 816 and the camera 820 to capture images and/or video of the eye(s) of the patient 806 during the administration of the vision screening test(s). For example, the radiation sensor(s) 816 may capture data indicative of reflected radiation from the eye(s) of the patient 806 during the activation of one or more of the radiation source(s) 814. The data may include grayscale image data and/or video data of the eye(s). The image capture control component 834 may synchronize the camera 820 to capture color image(s) and/or video data of the eye(s) with the activation of the white light source(s) 818 so that the eye(s) are illuminated by white light radiation during the capture of the color image and/or video data. In some examples, images of the left and the right eye may be captured under different illumination conditions (e.g., from a different individual source), so that the relative angle of illumination with the optical axis of the particular eye is the same for the left and the right eye. In other examples, images of both eyes may be captured simultaneously under the same illumination. As described herein, the image capture control component 834 of the vision screening device 804 may generate grayscale images of the eye(s) illuminated under NIR radiation, and color images of the eye(s) illuminated under white light. Capturing both the grayscale images and the color images may enable the detection of a wider range of diseases and abnormalities of the eyes.

In some examples, the computer-readable media 830 may also store a data analysis and visualization component 836. The data analysis and visualization component 836 may be configured to analyze the image and/or video data collected, detected, and/or otherwise captured by components of the vision screening device 804 (e.g., by the radiation sensor(s) 816, and the camera 820) during one or more vision screening tests. For example, the data analysis and visualization component 836 may analyze the data to determine location of the pupils of the eye(s) in the images, and identify a portion of the image(s) corresponding to the pupil (e.g., pupil image(s)). The data analysis and visualization component 836 may analyze the pupil image(s) to determine characterizations of appearance of the pupil(s) in the pupil image(s). For example, in the instance of the color image(s) captured by the camera 820, the characterizations may include an average color value, variance of color values, measure of uniformity, presence of inclusions, and the like. In the instance infrared image(s) captured by the radiation sensor(s) 816, the characterizations may include average grayscale value and variance of grayscale values, instead of the color, in addition to measures of uniformity and the presence of inclusions. The data analysis and visualization component 836 may further compare the left pupil image(s) and the right pupil image(s) to determine differences in appearance between the left and right pupils. For example, the differences may correspond to a difference in average color value or average grayscale value between the left pupil image(s) and right pupil image(s). The data analysis and visualization component 832 may also compare the pupil image(s) with standard pupil image(s) and/or pupil image(s) of the patient 806 captured during previous vision screening(s) to determine differences in appearance, such as differences in average color value or grayscale value, differences in the measure of uniformity, differences in detected inclusions, and the like. In any of the examples above, all captured image(s) or a subset of the captured grayscale and/or color images may be used to determine differences. In some examples, grayscale image(s) may not be used, and the difference may be determined based on the color image(s). It is to be noted that pixels of grayscale images may also be considered to have a color value, wherein the color value is determined by using the same grayscale value for each of the three-color channels (e.g., RGB). For example, a pixel with a grayscale value of 828, may be determined to have a color value of (128, 828, 828) in the RGB color space. The data analysis and visualization component 836 may also apply additional image processing steps to the grayscale image(s) and/or the color image(s) which may improve detection of disease states. For example, images may be sharpened, specific colors may be boosted or attenuated, color or brightness of the images may be balanced, and the like.

Further, the data analysis and visualization component 836 may be configured to receive, access, and/or analyze standard data associated with vision screening. For example, the data analysis and visualization component 836 may be configured to access or receive data from one or more additional databases (e.g., the screening database 844, a third-party database, etc.) storing testing data, measurements, and/or values indicating various thresholds or ranges within which measured values should lie. Such thresholds or ranges may be associated with patients having normal vision health and may be learned or otherwise determined from standard testing. The data analysis component and visualization component 836 may utilize the standard data for comparison with the average values and differences determined during the vision screening test(s) as described above. For example, the standard data may indicate a threshold or a range for a difference between color values of the left and right pupil images, where a difference greater than the threshold, or outside the range, corresponds to an abnormality in the eye(s) of the patient. Alternatively, or in addition, the data analysis and visualization component 836 may access a previous vision screening of the patient 806 and compare the values and differences with corresponding data from the previous screening(s). For example, an average color value of the pupil may be compared with an average color value from a previous screening to determine a difference. This difference may then be compared with standard thresholds or ranges to determine presence of an abnormality, as described above. Separate threshold(s) and/or range(s) may be indicated in the standard data for different types of diseases and abnormalities. In addition, the threshold(s) and/or range(s) associated with the vision screening test may also be based on the testing category of the patient 806 (e.g., the age group or medical history of the patient 806), where the threshold(s) and/or range(s) may be different for different testing categories. The data analysis and visualization component 836 may store as a part of the patient data 840, images and/or video captured or generated during the vision screening test(s), measurements associated with the vision screening test(s), test results, and other data in a database(e.g., in the screening database 844) for comparison of data over time to monitor vision health status and changes in vision health. In some examples, the stored images may include images of the face or partial face (e.g., eyes and part of nose) of the patient 806.

Based on the comparison with a threshold and/or range described above, the data analysis and visualization component 836 may generate a normal/abnormal or a pass/refer determination for each of the eyes of the patient 806. For example, if all values and differences measured are less than on equal to corresponding threshold(s), or fall within the corresponding range(s) of the standard data, a “normal” or “pass” determination may be made by the data analysis and visualization component 836, and an “abnormal” or “refer” determination made otherwise to indicate a referral for further screening. Alternatively, or in addition, the data analysis and visualization component 836 may generate a normal/abnormal determination for each of the diseases and/or abnormalities screened for during the vision screening session.

In examples, the data analysis and visualization component 836 may utilize one or more machine learning techniques to generate a diagnosis of specific diseases and/or types of abnormalities. For example, machine learning (ML) models may be trained with normal images of eyes, and images of eyes labeled as exhibiting various disease conditions and abnormalities. The trained ML model(s) may then generate an output indicating a disease or abnormality diagnosis when provided, as input, an image of the eye captured during the vision screening of the patient 806. In such examples, the data analysis and visualization component 836 may directly generate the output by providing an image of the eye as input to the trained ML model(s), without computing differences between pupil images or applying comparisons with a threshold and/or range. In some examples, a plurality of trained ML model(s) may be used, each ML model being trained to detect a specific disease or abnormality. In such examples, each ML model outputs a binary present/absent indication to indicate if the input image exhibits the disease or abnormality that the ML model is trained to detect. The data analysis and visualization component 836 may provide an image of the eye as input to each ML model of the plurality of trained ML model(s) for detecting one or more of a plurality of diseases and abnormalities. In examples, the ML models may be neural networks, including convolutional neural networks (CNNs). In other examples, the ML models can also include regression algorithms, decision tree algorithms, Bayesian classification algorithms, clustering algorithms, support vector machines (SVMs) and the like.

The data analysis and visualization component 836 may also generate visualizations of the eye(s) using the image(s) and/or video data captured by the radiation sensor(s) 816 and/or the camera 820. For example, a first visualization may include a composite image of the eye(s) incorporating both color information from the color image(s) and grayscale information from the grayscale image(s) captured by the radiation sensor(s) 816 under NIR illumination. The generation of the first visualization may include detection and identification of structures of the eye(s) such as pupils and/or lenses, followed by registration of the grayscale image(s) and the color image(s) so that the pupils are located in the same position in both types of image(s). The composite image may then be generated by using grayscale pixel values from the grayscale image(s) in some portions of the composite image and color pixel values from the color image(s) in other portions of the composite image. The portions of the composite image using grayscale pixel values and the portions using color pixel values may correspond to areas depicting different structures of the eye(s) (e.g., fovea, retina, cornea etc.). The composite image may more clearly delineate structures of the eye(s) for improved detection and assessment of diseases and/or abnormalities of the eye(s).

In another example, a second visualization may include a sequence of still images, or an animated video comprising the sequence of still images. In some instances, the sequence of images may be captured by the radiation sensor(s) 816 or the camera 820 while the eye(s) are illuminated by the radiation source(s) 814 or white light source(s) 818 at a progression of different angles along different axes with respect to the optical axis. In some examples, the visualization may include graphics and/or color-coding indicative of areas of the image of the eye(s) that are flagged as being abnormal. As described herein, the data analysis and visualization component 836 of the vision screening device 804 may process the grayscale images and the color images of the eye(s) captured during the administration of the vision screening test(s), to determine diseases and/or abnormalities associated with the eye(s) of the patient. In addition, the data analysis and visualization component 836 may generate, based on the grayscale and color images, visualizations of the eye(s) that aid a clinician or an operator of the vision screening device 804 to identify diseases and/or abnormalities of the eye(s).

The computer-readable media 830 may additionally store an output generation component 838. The output generation component 838 may be configured to receive, access, and/or analyze data from the data analysis and visualization component 836, and generate the output 812. For example, the output generation component 838 may utilize the normal/abnormal determinations of the data analysis and visualization component 836 to generate a recommendation in the output 812. The recommendation may indicate whether the screening results of the patient 806 indicate normal eye health, or further screening is needed based on one or more of the screening tests resulting in an “abnormal” finding. In addition, the output generation component 838 may incorporate all or a subset of the visualizations generated by the data analysis and visualization component 836 into the output 812 for aiding in diagnosis of the condition of the eye(s). Portions of the images and/or video captured by the radiation sensor(s) 816 or the camera 820 may also be included in the output 812. Additionally, if abnormality is determined, the output generation component 838 may incorporate a likely diagnosis into the output 812 based on the analysis by the data analysis and visualization component 836. The output 812 may be presented to the operator of the device via an interface of the device (e.g., on the display screen 822 of the vision screening device 804). In examples, the operator display screen may not be visible to the patient, e.g., the operator display screen may be facing in a direction opposite the patient. The output generation component 838 may also store the output 812, which may include a recommendation, diagnosis, measurements, captured images/video and/or the generated visualizations in a database, such as the screening database 844, for evaluation by a clinician, or for access during subsequent vision screening(s) of the patient 806. The screening database 844 may provide access to authorized medical professionals to enable printing of reports or further assessment of the data related to the screening of the patient 806.

Although FIG. 8 illustrates example processor(s) 828 and computer-readable media 830 storing a patient screening component 832, an image capture control component 834, a data analysis and visualization component 836, an output generation component 838 and/or other components and/or other items as components of the vision screening device 804, in any of the examples described herein, the vision screening system 810 may include similar components and/or the same components. In such examples, the vision screening system 810 may include processor(s) 846 and computer-readable memory 848 that are configured to perform the functions of some or all of the components in the computer-readable memory 830 of the vision screening device 804. For example, one or more of the components of the computer-readable memory 830 may be included in analysis component(s) 850 of computer-readable memory 848 and be executable by the processor(s) 846. In such examples, the vision screening system 810 may communicate with the vision screening device 804 using network interface(s) 852, and via the network 808, to receive data from the vision screening device 804 and send results (e.g., output 812), back to the vision screening device 804. The vision screening system 810 may be implemented on a computer proximate the vision screening device 804 or may be at a remote location. For example, the vision screening system 810 may be implemented as a cloud service on a remote cloud server.

The network interface(s) 852 may enable wired and/or wireless communications between the components and/or devices shown in system 800 and/or with one or more other remote systems, as well as other networked devices. For instance, at least some of the network interface(s) 852 may include a personal area network component to enable communications over one or more short-range wireless communication channels. Furthermore, at least some of the network interface(s) 852 may include a wide area network component to enable communication over a wide area network. Such network interface(s) 852 may enable, for example, communication between the vision screening system 810 and the vision screening device 804 and/or other components of the system 800, via the network 808. For instance, the network interface(s) 852 may be configured to connect to external databases (e.g., the screening database 844) to receive, access, and/or send screening data using wireless connections. Wireless connections can include cellular network connections and connections made using protocols such as 802.11a, b, g, and/or ac. In other examples, a wireless connection can be accomplished directly between the vision screening device 804 and an external system using one or more wireless protocols, such as Bluetooth, Wi-Fi Direct, radio-frequency identification (RFID), infrared signals, and/or Zigbee. Other configurations are possible. The communication of data to an external database can enable report printing or further assessment of the patient's visual test data. For example, data collected and corresponding test results may be wirelessly transmitted and stored in a remote database accessible by authorized medical professionals.

It should be understood that, while FIG. 8 depicts the system 800 as including a single vision screening system, in additional examples, the system 800 may include any number of local or remote vision screening systems substantially similar to the vision screening system 810 and configured to operate independently and/or in combination and configured to communicate via the network 808.

As discussed herein, FIG. 8 depicts a vision screening device 804 that includes components for administering vision screening tests to a patient. In some examples, one or more components may be implemented on a remote vision screening system communicating with the vision screening device 804 over a network 808.

FIG. 9 illustrates a method 900 for operating the vision screening device, in accordance with one or more examples. The example vision screening device may include one or more of the same components included in the vision screening devices as described herein. In some additional examples, the vision screening device can include different components that provide similar functions to the system 800 or other vision screening devices described herein.

FIG. 9 provides a flow diagram illustrating an example method for vision screening, as described herein. The method in FIG. 9 is illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the method illustrated in FIG. 9. In some embodiments, one or more blocks of the method illustrated in FIG. 9 can be omitted entirely.

The operations described below with respect to the methods illustrated in FIG. 9 can be performed by any of the devices or systems described above and/or by various components thereof.

With reference to the method 900 illustrated in FIG. 9, at operation 902, the vision screening device determines a distance to the patient using an autofocus sensor and/or distance sensor such as a range finder or other suitable component used for determining a distance between objects. The sensor may be used to identify a distance between the patient and the vision screening device. After determining the distance to the patient, the vision screening device may display the distance on a clinician-facing display and may instruct the clinician to adjust the position of the vision screening device and/or patient to reach a predetermined distance (e.g., five or ten feet as discussed herein). Upon reaching the predetermined distance, the vision screening device may automatically display one or more potential exams to perform using the device.

At operation 904, the method 900 includes displaying a patient exam. The patient exam may be selected and may include one or more different exams that may be performed simultaneously and/or sequentially. The patient exam may include symbols or digital objects displayed on a user-facing display of the vision screening device. Accordingly, the vision screening device may present such digital objects as required or requested for a particular exam.

At operation 906, the method 900 includes the image capture control component 834 and/or one or more processors associated therewith may cause a radiation source to emit radiation (e.g., near-infrared (NIR) radiation). For example, the radiation source may comprise NIR LEDs of the radiation source(s) 814 of the vision screening device 804, configured to emit NIR radiation during a period of time corresponding at least in part to the administration of a vision screening test indicated for the patient by the patient screening component 832. In some examples, the image capture control component 834 may cause radiation of different wavelengths to be emitted e.g., a first radiation source may emit radiation of a first wavelength, and a second radiation source may emit radiation of a second wavelength. The image capture control component 834 may activate LEDs of the radiation source(s) 814 individually or in groups to produce radiation impinging on the eyes at different angles relative to the optical axis of the eye. In addition, the image capture control component 834 may activate different wavelengths of radiation combined with different angles of incidence on the eyes. In some examples, the arrangement of the pattern of activation of the NIR LEDs allows for different illumination patterns to be presented to the eye(s) of the patient 806, and refractive error of the eye(s) may be accurately measured based on images captured under the illumination pattern selected. Additional details regarding illumination patterns used in examination protocols for determining refractive error can be found in U.S. patent application Ser. No. 9,237,846, referred to above and incorporated herein by reference.

At operation 908, the method 900 includes redirecting reflected radiation using a beam splitter. The received or incident reflected light and/or radiation is reflected using a beam splitter into a lens and filter assembly that may further filter and/or transform the received light to a suitable form for consumption by a sensor of the vision screening device, such as a sensor capable of detecting both visible and NIR light.

At operation 910, the image capture control component 834 may cause a sensor of the vision screening device (e.g., radiation sensor(s) 816 of the vision screening device 804), to capture radiation reflected by the eye(s) of the patient under illumination by the radiation source(s) 814. The image capture control component 834 may receive data indicative of the radiation captured by the radiation sensor(s) 816. The data may include grayscale image(s) and or video of the eye(s) illuminated by radiation from different angles as described above. For example, the image capture control component 834 may cause a sensor to capture a first image under illumination from a first set of NIR LEDs, and a second image under illumination from a second set of NIR LEDs. In some examples, near-infrared may be a first wavelength band emitted by a first radiation source(s), and the image capture control component 834 may further activate a second radiation source(s) emitting radiation in a second wavelength band, and cause a sensor to capture a third image under illumination from the second radiation source. In addition, the image capture control component 834 may cause the sensor(s) to capture images of both eyes simultaneously, or one eye at a time. For example, the image capture control component 834 may change the activation of the illumination source(s) (e.g., activate different LEDs of the radiation source(s) 814, 818) after capturing an image of the left eye and before capturing an image of the right eye, so that both the left and right eye are illuminated from a same angle relative to the eye during the image capture.

At operation 912, the image capture control component 834 and/or one or more processors associated therewith may cause a white light source (e.g., white light source(s) 818 of the vision screening device 804), to emit white light to illuminate the patient during a period of time, and during at least a part of the administration of the vision screening test. Similar to the radiation source(s) described at operation 602, individual white light sources of the white light source(s) 818 may also be activated to illuminate the eye from different angles relative to the optical axis.

At operation 914, the image capture control component 834 may cause a camera (e.g., the camera 820 of the vision screening device 804), to capture color image(s) of the eye(s) of the patient while under white light illumination. In some examples, the image capture control component 834 may also cause the camera to capture video data. The image capture control component 834 may store the color image(s) and/or video in a database for review by a clinician. In addition, the image capture control component 834 may cause the camera to capture a color image of the patient's face and store the image in the patient data 840 as a photo identifier of the patient. The data analysis and visualization component 836 may utilize the color image(s) to generate a composite image, and determine differences between the images.

At operation 916, the data analysis and visualization component 836 may determine one or more test results corresponding to a test being performed using the vision screening device and generate an output of the test results. The test results may be determined by components of the vision screening device and/or a connected system and the output may be provided.

As discussed, the example method 900 may be performed by the components of the vision screening device 804 executed by the processor(s) 828 of the vision screening device 804. The example method 900 illustrates operations performed during at least a part of a vision screening test administered to a patient (e.g., the patient 806) to determine diseases and/or abnormalities associated with the eye(s) of the patient based on images of the eye(s) captured under illumination of different wavelengths. In alternative examples, some or all of the operations of method 900 may be executed by processor(s) 846 of a vision screening system 810 that is connected to the vision screening device 804 via network 808.

Based at least on the description herein, it is understood that the vision screening devices and associated systems and methods of the present disclosure may be used to assist in performing one or more vision screening tests, including test(s) to screen for diseases and/or abnormalities of the eye(s) of the patient. The components of the vision screening device described herein may be configured to generate radiation of different wavelengths, in addition to white light, to illuminate the eyes of the patient undergoing vision screening, capture image(s) of the eye(s) under different illumination conditions, generate visualizations that aid in the diagnosis of disease conditions, determine differences between pupil images, and determine an output indicating a diagnosis, recommendation or results of the screening test. An exemplary vision screening device may include radiation source(s) for generating radiation of different wavelengths, and sensor(s) for capturing the reflected radiation from the eye(s) of the patient, a white light source and a camera configured to capture color image(s) of the eye(s) of the patient under white light illumination, and display screen(s) for displaying the output to an operator of the vision screening device. The device described herein may be used for screening a patient for ocular diseases and abnormalities without requiring inputs or feedback from the patient, and without requiring dilation of the eyes, thereby allowing the device to be used for screening very young, very old, incapacitated, or uncooperative patients.

The foregoing is merely illustrative of the principles of this disclosure and various modifications can be made by those skilled in the art without departing from the scope of this disclosure. The above-described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A vision screening device, comprising:
a radiation source comprising radiation of a first wavelength and visible light emitted substantially parallel with a first optical axis;
a first sensor configured to capture radiation reflected by an eye of a patient and further configured to capture a color image of the eye of the patient;
a second sensor;
a first display disposed on a patient-facing side of the vision screening device;
a second display disposed on a user-facing side of the vision screening device opposite the patient-facing side;
one or more processors; and
one or more non-transitory computer-readable media having instructions stored thereon that, when executed by the processor cause one or more processors to:
determine, based on data from the second sensor, a distance to the patient;
display, via the first display, one or more digital objects for a vision test;
cause the radiation source to emit radiation of the first wavelength towards the patient;
cause the first sensor to capture first sensor data associated with a portion of the radiation reflected by the eye of the patient;
cause the white light source to illuminate the eye of the patient;
cause the first sensor to capture second sensor data associated with a portion of white light reflected by the eye of the patient; and
determine, based at least in part on the first sensor data and the second sensor data, an output indicative of a condition associated with the eye.

2. The vision screening device of claim 1, wherein the instructions are further configured to cause the one or more processors to provide, via the second display, directions to reach a predetermined distance to the patient.

3. The vision screening device of claim 1, further comprising a beam splitter positioned adjacent the patient-facing side, the beam splitter configured to direct reflected radiation towards the first sensor along a second optical axis, the second optical axis substantially perpendicular to the first optical axis.

4. The vision screening device of claim 3, further comprising an emitter board disposed within a housing of the vision screening device, the emitter board comprising the radiation source and configured to emit the radiation through the beam splitter and out of the housing.

5. The vision screening device of claim 4, wherein the emitter board is a first emitter board and the vision screening device further comprises a second emitter board disposed between the beam splitter and an emission surface of the vision screening device, the second emitter board further comprising a second radiation source and defining an opening configured to enable the radiation to emit outwards and to enable reflected radiation to pass through and reach the beam splitter.

6. The vision screening device of claim 4, wherein the emitter board comprises the white light source and the radiation source.

7. The vision screening device of claim 1, wherein the one or more processors being configured to provide the directions comprises determining an evaluation distance for one or more vision screening tests and instructing, via the first display, movement of the vision screening device to a position within a threshold distance of the evaluation distance.

8. A vision screening device, comprising:
a housing having a patient-facing side and a user-facing side;
a radiation source configured to emit radiation of a first wavelength out of an emission surface of the patient-facing side;
a white light source configured to emit white light out of the emission surface;
a first sensor configured to capture radiation reflected by an eye of a patient and further configured to capture a color image of the eye of the patient;
a second sensor configured to determine distance data representing a distance to a patient;
a first display disposed on the patient-facing side and configured to output characters for one or more vision screening tests; and
a second display disposed on the user-facing side and configured to output distance data and output data representative of results of the one or more vision screening tests.

9. The vision screening device of claim 8, wherein the radiation source, white light source, and first sensor are secured to a rigid chassis, the rigid chassis mounted within the housing using one or more shock absorbing components.

10. The vision screening device of claim 8, further comprising a beam splitter positioned between the radiation source and the emission surface, the beam splitter positioned to enable the radiation from the radiation source to exit the housing at the emission surface along a first direction and to reflect received radiation reflected from the patient along a second direction opposite the first direction towards the first sensor.

11. The vision screening device of claim 10, wherein an optical axis of the first sensor is perpendicular to an emission axis of the radiation source and the white light source, the emission axis extending outwards from the emission surface to the patient.

12. The vision screening device of claim 10, further comprising an emitter board including the radiation source and the white light source.

13. The vision screening device of claim 12, wherein the emitter board is disposed within the housing with the beam splitter positioned between the emitter board and the emission surface.

14. The vision screening device of claim 12, further comprising a second emitter board disposed between the beam splitter and the emission surface, the second emitter board further comprising a second radiation source and defining an opening configured to enable the radiation to emit outwards and to enable reflected radiation to pass through and reach the beam splitter.

15. The vision screening device of claim 10, further comprising a polarizing filter configured to polarize reflected radiation from the patient received at the emission surface.

16. A system, comprising:
a housing having a patient-facing side and a user-facing side;
a radiation source configured to emit radiation of a first wavelength out of an emission surface of the patient-facing side;
a white light source configured to emit white light out of the emission surface;
a first sensor configured to capture radiation reflected by an eye of a patient and further configured to capture a color image of the eye of the patient;
a second sensor configured to determine distance data representing a distance to a patient;
a first display disposed on the patient-facing side and configured to output characters for one or more vision screening tests; and
a second display disposed on the user-facing side and configured to output distance data and output data representative of results of the one or more vision screening tests.

17. The system of claim 16, further comprising:
one or more processors; and
one or more non-transitory computer-readable media having instructions stored thereon that, when executed by the processor cause one or more processors to:
determine a distance to the patient;
provide, via the second display, directions to reach a predetermined distance to the patient;
display, via the first display, one or more digital objects for a vision test;
cause the radiation source to emit radiation of the first wavelength towards the patient;
cause the first sensor to capture first sensor data associated with a portion of the radiation reflected by the eye of the patient;
cause the white light source to illuminate the eye of the patient;
cause the first sensor to capture second sensor data associated with a portion of the white light reflected by the eye of the patient; and
determine, based at least in part on the first sensor data and the second sensor data, an output indicative of a condition associated with the eye.

18. The system of claim 16, further comprising a beam splitter positioned between the radiation source and the emission surface, the beam splitter positioned to enable the radiation from the radiation source to exit the housing at the emission surface along a first direction and to reflect received radiation reflected from the patient along a second direction opposite the first direction towards the first sensor.

19. The system of claim 18, further comprising an emitter board comprising the radiation source and white light source disposed within the housing with the beam splitter positioned between the emitter board and the emission surface.

20. The system of claim 19, further comprising a second emitter board disposed between the beam splitter and the emission surface, the second emitter board further comprising a second radiation source and defining an opening configured to enable the radiation to emit outwards and to enable reflected radiation to pass through and reach the beam splitter.
